(19) United States
(12) Patent Application Publication
(10) Pub. No.: US 2019/0298209 A1
(43) Pub. Date: Oct. 3, 2019

(54) HEARTBEAT DETECTION

(71) Applicant: Livmor, Inc., Irvine, CA (US)

(72) Inventors: Ken Persen, Irvine, CA (US); Ross Grady Baker, JR., Irvine, CA (US); Lucy Zhuang, Irvine, CA (US)

(73) Assignee: Livmor, Inc., Irvine, CA (US)

(21) Appl. No.: 16/368,473

(22) Filed: Mar. 28, 2019

Related U.S. Application Data
(60) Provisional application No. 62/649,527, filed on Mar. 28, 2018.

(51) Int. Cl. A61B 5/0464; A61B 5/024; A61B 5/00

(52) U.S. Cl. CPC A61B 5/0464; A61B 5/02416; A61B 5/725; A61B 5/0002; A61B 5/681; A61B 5/02438

(57) ABSTRACT

A photoplethysmographic (PPG) signal may be received as communicated by a PPG sensor of a wearable device worn by a subject. A first heartbeat and a second heartbeat may be determined from a maximum gradient of the PPG-signal. A heart rate may be determined based on at least the first heartbeat and the second heartbeat and may be transmitted to at least the wearable device.

### RELATED APPLICATION(S)

This application claims priority to and the benefit of U.S. Provisional Application No. 62/649,527, filed Mar. 28, 2018, titled “Systems and Methods for Heartbeat Detection,” which is hereby incorporated by reference.

### DESCRIPTION OF THE RELATED ART

Electrical and physiological characteristics of the human heart can be measured using, for example, sensors such as electrocardiogram (ECG) sensors or photoplethysmograph (PPG) sensors. Signals from such sensors may then be analyzed to determine useful and informative health states of a patient, such as heart rates, particular heart rhythms, and the like.

### SUMMARY

Systems, methods, and computer software are disclosed that may be used to detect heartbeats and determine a heart rate. For example, a photoplethysmographic (PPG) signal is received as communicated by a PPG sensor of a wearable device worn by a subject. A first heartbeat and a second heartbeat are determined from a maximum gradient of the PPG-signal. A heart rate is determined based on at least the first heartbeat and the second heartbeat. The heart rate is transmitted to at least the wearable device.

In some implementations, determining the first heartbeat further includes utilizing the maximum gradient, at least two other data points from a gradient of the PPG-signal, and a mathematical method. The two other data points may be a first PPG gradient and a second PPG gradient and the mathematical method may be spline interpolation. In some implementations, a state based sequence detector may be utilized to determine the two other data points from the gradient of the PPG-signal and to implement the mathematical method.

The present disclosure also describes receiving a photoplethysmographic (PPG) signal communicated by a PPG sensor of a wearable device worn by a subject and determining a first PPG-signal peak and a second PPG-signal peak from at least a portion of the PPG-signal. A first heartbeat and a second heartbeat are from the first PPG-signal peak and the second PPG-signal peak. A heart rate is determined based on at least the first heartbeat and the second heartbeat and transmitted to at least the wearable device.

In some implementations, determining the first heartbeat further includes utilizing the first PPG-signal peak, at least two other data points from the PPG-signal, and a mathematical method. The two other data points from the PPG-signal may be a positive-going zero crossing and a negative-going zero crossing nearest the PPG-signal peak. The mathematical method may includes spline interpolation. In some implementations, a state based sequence detector is utilized to determine the two other data points from the PPG-signal and to implement the mathematical method.

Also disclosed is receiving a photoplethysmographic (PPG) signal communicated by a PPG sensor of a wearable device worn by a subject. At least a portion of the PPG-signal may be processed through frequency band filters to create band outputs corresponding to heartbeat interval ranges. An indicated band output among the band outputs may be utilized to determine a first heartbeat and a second heartbeat. A heart rate may be determined based on at least the first heartbeat and the second heartbeat and transmitted to at least the wearable device.

In some implementations, the heartbeat interval ranges correspond to at least a normal range, a tachycardia range, and a bradycardia range. A bandwidth of at least one of the frequency band filters is chosen to prevent inclusion of two or more successive harmonics of the PPG-signal. In some implementations, processing the portion of the PPG-signal through the frequency band filters further includes utilizing successively increasing semi-dyadic cascade of low-pass/high-pass separations. Also, amplitudes for the band outputs can be determined as well as determining a largest amplitude band output among the band outputs. The indicated band output may correspond to the largest amplitude band output.

In yet other implementations, determining the first heartbeat may include determining a first PPG-signal peak of the indicated band output. The first heartbeat may be determined utilizing the first PPG-signal peak, at least two other data points from the PPG-signal, and a mathematical method. The two other data points may include a positive-going zero crossing and a negative-going zero crossing nearest to the first PPG-signal peak. Also, a state based sequence detector may be utilized to determine the two other data points from the PPG-signal and to implement the mathematical method. The mathematical method may be spline interpolation. In some implementations, a second largest amplitude band output may be determined and the second largest amplitude band output may be utilized to determine an anticipated heartbeat.

Determining the anticipated heartbeat can include determining a PPG-signal peak of the second largest amplitude band output and also utilizing the PPG-signal peak of the second largest amplitude band output, at least two other data points from the second largest amplitude band output, and a mathematical method. The two other data points may comprise a positive-going zero crossing and a negative-going zero crossing nearest to the PPG-signal peak of the second largest amplitude band output. The mathematical method may be spline interpolation. Also, the first anticipated heartbeat may be utilized in determining the heart rate when the indicated band output changes, to provide faster heart rate tracking and transmission to the wearable device.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also contemplated that may include one or more processors and one or more memories coupled to the one or more processors. A memory, which can include a computer-readable storage medium, may include, encode, store, or the like, one or more programs that cause one or more processors to perform one or more of the operations described herein. Computer implemented methods consistent with one or more implementations of the current subject matter can be implemented by one or more data processors residing in a single computing system or across multiple computing systems. Such multiple computing systems can be connected and can exchange data and/or commands or other instructions or the like via one or more connections, including but not limited to a connection over a network (e.g., the internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to particular implementations, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations.

FIG. 1 illustrates an exemplary system that can provide for the monitoring of user health characteristics and provide health-related guidance.

FIG. 2 illustrates an implementation of a user wearable device.

FIG. 3 illustrates an implementation of a communication device.

FIG. 4 illustrates an implementation of a server.

FIG. 5 illustrates an exemplary PPG-signal and corresponding PPG-signal gradient.

FIG. 6 illustrates an exemplary implementation of a heartbeat determination method.

### DETAILED DESCRIPTION

The subject matter described herein relates systems, methods and software for monitoring the heath of a user and providing the user with health guidance.

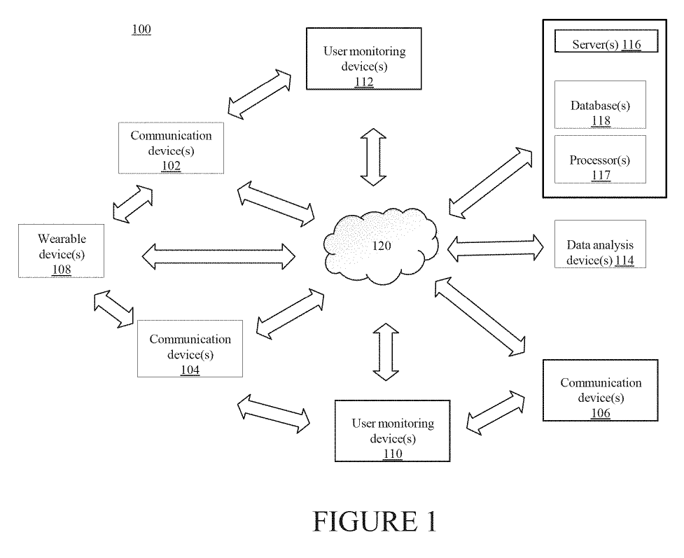

FIG. 1 illustrates an exemplary system 100 that can provide for the monitoring of health characteristics of a user (for example, a human patient, or other living organism) and can provide health guidance to the user based on the health characteristics monitoring.

In some implementations, the exemplary system 100 depicted in FIG. 1 may include elements such as user wearable device(s) 108 (e.g., a smart watch), communication devices 102, 104, and 106 (e.g., a mobile phone or PC), user monitoring devices 110 and 112 (e.g., a separate smart scale or blood glucose monitor), data analysis device(s) 114, server(s) 116 (e.g., including processor(s) 117 and database(s) 118), and network(s) 120. The server(s) 116 and devices illustrated in FIG. 1 may include communication lines or ports to enable the exchange of information within a network (e.g., network 120), or within other computing platforms via wired or wireless techniques (e.g., Ethernet, fiber optics, coaxial cable, WiFi, Bluetooth, near field communication, or other technologies).

It should be noted that, while one or more operations are described herein as being performed by particular components of system 100, those operations may, in some embodiments, be performed by other components of system 100. As an example, while one or more operations are described herein as being performed by components of data analysis device(s) 114, those operations may, in other embodiments, be performed by components of the user wearable device(s) 108, by components of the communications devices 102, 104, and 106, and/or by other components of system 100.

The user wearable device(s) 108 may be a smart watch (for example, Samsung's Gear, the Apple Watch, etc.), or any other device that a user can wear. A user wearable device 108 may include one or more sensors that are integrated within the device. For example, a user wearable device 108 that is a smart watch may include motion sensors (for example, accelerometers), bio-impedance sensors, ECG sensors, ballistocardiogram sensors, acoustic sensors (for example, ultrasound), photo plethysmograph (PPG) sensors that use light-based technology to sense a rate of blood flow, and other sensors. Wearable device 108 may also be considered herein to include sensors that are worn on a user's body but not integrated within the main wearable portion (for example, an ECG sensor worn on a user's chest that is not integrated with a smart watch, but which nevertheless communicates with the smart watch).

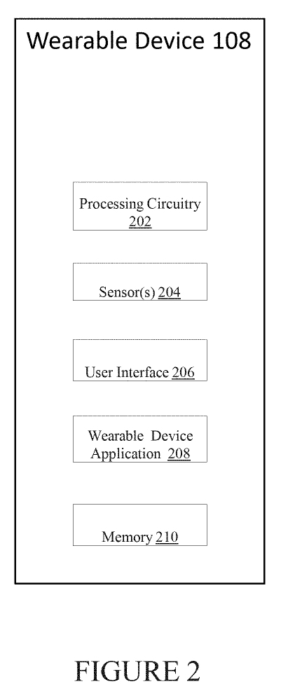

FIG. 2 illustrates wearable device 108, including processing circuitry 202, sensor(s) 204, wearable user interface 206, wearable device application 208, and memory 210. As noted, sensor(s) 204 may include multiple sensors integrated with the main wearable portion of the device and/or sensors located elsewhere on the user's body. The wearable device application 208, and signals from sensor(s) 204, may be stored in memory 210.

A user may interact with wearable user interface 206, for example, to enter data such as age, height, weight and gender, or to view measured or calculated metrics such as heart rate, pulse rate variability, stress level, breathing guidance, and the like.

Wearable device application 208 may run on processing circuitry 202 and perform such operations as receiving signals from sensor(s) 204, calculating various health characteristics, outputting the display of information, providing health guidance to the user, etc.

Wearable device 108 may undergo certain configurations during a calibration period. For example, a user may wear device 108 for a 24-hour calibration period upon first use to allow for the collection of user information from sensor(s) 204. For example, the collection of characteristics such as pulse rate or respiration rate over a period of time may facilitate device calibration and provide user information helpful in the future analysis of signals and the provision of health guidance to the user. In some beneficial implementations, the calibration may be performed while the user is wearing a single lead ECG or other sensor(s) for reference purposes.

Communication devices 102, 104, and 106 may include any type of mobile or fixed device, for example, a desktop computer, a notebook computer, a smartphone, a tablet, or other communication device. Users may, for instance, utilize one or more communication devices 102, 104, and 106 to interact with one another, with one or more wearable devices, one or more servers, or other components of system 100.

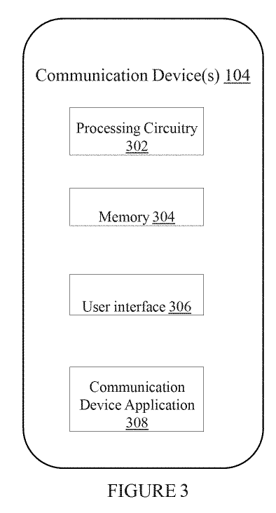

FIG. 3 illustrates some components of an exemplary communication device 104, including processing circuitry 302, memory 304, user interface 306, and communication device application 308. The processing circuitry 302, memory 304, and user interface 306 function similarly to the processing circuitry 202, memory 210, and user interface 206, respectively, in FIG. 2, although the application and user interface of a communication device will commonly have greater functionality than that of a wearable device.

In some implementations, communication device application 308 may be a mobile application (for example, a smartphone application), or a web application. The communication device application 308, in some implementations, can communicate with the user wearable device application 208 via Bluetooth (or any other method of wired or wireless communication) and/or may transmit measurements for archival and post-processing to a cloud-based database (for example, database(s) 118). The communication device application 308 may aggregate data from the other sensors (for example, from user monitoring devices 110 and 112), perform pre-transmission processing locally, and transmit data for further processing or viewing.

In some implementations, user monitoring devices 110 and 112 may include a blood pressure monitoring device (for example, a blood pressure cuff), a weight monitoring device (for example, a scale), a blood glucose monitoring device, etc. User monitoring devices 110 and 112 may measure health states of the user different from the health states measured by user wearable device(s) 108.

The health monitoring and guidance systems and methods detailed herein typically utilize signals coming from one or more sensors that may be in contact with a user's body and that are sensing information relevant to the user. Sensors can be integrated with a wearable device, communicating with a wearable device, or can instead be separate from a wearable device and communicating with system 100 through other components.

As discussed further herein, system 100 can include components and methods for acquiring particular signals, for processing such signals (e.g., noise reduction), and for modifying signal acquisition methods. Each of these activities may be performed by any of the components of system 100.

In one implementation, a user wearable device 108 may capture an optical signal (for example, a pulse signal) from optical sensor(s) utilizing green and/or an infrared wavelengths. Wearable device 108 may also capture a motion signal that could be used to assess noise or interference resulting from motion of a user wearing device 108 or to assess other parameters relevant to health analysis and guidance.

In some implementations, the optical signal and the motion signal can be buffered within a memory (for example, memory 210 in FIG. 2) of the user wearable device(s) 108 for a predetermined time period, and the optical signal and the motion signal can then be provided to other processors for the processing of these signals (for example, processing circuitry 302 of communication device 104 in FIG. 3 or the circuitry of data analysis device(s) 114). As such, power consumption of the user wearable device(s) 108 may be conserved or optimized. Alternatively, in some implementations, processing circuitry 202 of a user wearable device 108 may be used for processing of the optical pulse signal and the motion signal captured by the user wearable device 108.

Signal collection or acquisition from an optical sensor at a 12-50 Hz sampling frequency may be used, especially in optimal conditions such as the general absence of user motion combined with low-levels of perfusion and low ambient light interference. Various conditions can affect the sampling rate considered optimal, but one of the most impactful conditions is the motion of a user wearing the device 108.

Signal processing challenges caused by user motion may be overcome by adjusting various parameters relating to signal acquisition. For example, optical sensor performance can be adjusted when activity is detected by a motion sensor (e.g., a three-axis accelerometer). In some implementations, if motion above a specific threshold is detected, any or all of the following acquisition parameters of an optical sensor can be adjusted to overcome the level of noise and to improve the accuracy of health characteristic determination: (i) sampling frequency, (ii) LED power, and/or (iii) pulses per sample. Conversely, in some implementations, if motion below a specific threshold is detected, then each of these acquisition parameters may be adjusted to maintain a specific level of performance and measurement precision, while also conserving power.

In the general absence of user motion, a sampling frequency of approximately 20 Hz may be appropriate but, in more challenging environments, sampling can be increased to 100 or 200 Hz or, if necessary, up to 1000 Hz or more to ensure that signals are received that are useful for the analysis of user health characteristics. The use of other sampling frequencies is also contemplated.

Various health characteristics of a user may be determined utilizing signals from the sensors discussed herein. As one example, sensors associated with a user wearable device, such as a watch, may be utilized to determine a user's heart rate, pulse rate variability (PRV) or heart rate variability (HRV). Heart rate is typically described as the number of heart beats per minute, while HRV and PRV both refer to the variability of the time intervals between beats. HRV typically refers to variability measurements based on electrocardiography and can be derived from R-R intervals in the standard PQRS waveform. An HRV determination may utilize an ECG sensor on a user that may communicate with wearable device 108. PRV, on the other hand, typically refers to variability determinations based on sensors placed proximal to peripheral arteries, such as optical sensor(s) on a user's wrist that provide a peripheral pulse waveform absent of the morphology information seen in an ECG signal.

User health characteristics may be determined through signal analysis performed on user wearable device 108 or other components of system 100 such as communication device 102 or data analysis device 114, or the analysis may be performed on more than one component of system 100.

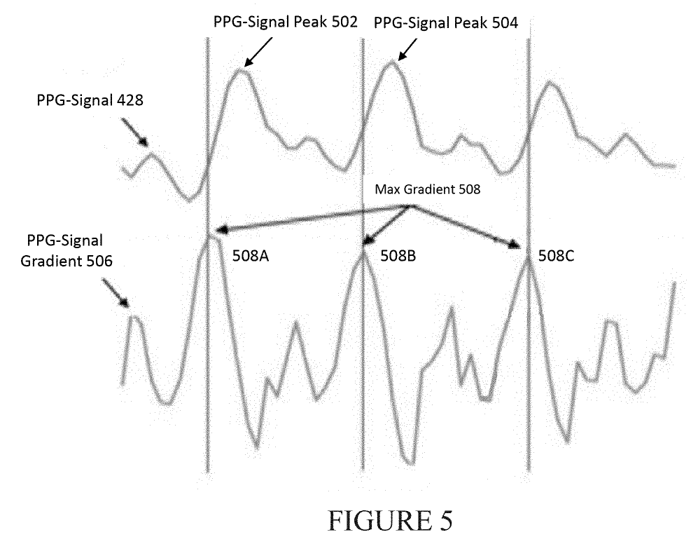

In some implementations, the received sensor signal can be an ECG signal, and the time at which each heartbeat has occurred can be determined, for example, from each R spike in the ECG waveform. Alternatively, the time at which each heartbeat has occurred may be determined from a PPG-signal. In one exemplary implementation, heartbeat times from a PPG-signal may be determined utilizing maximum points of a PPG gradient plot (see, e.g., FIG. 5). Improved resolution for such determinations may be obtained through a variety of methods, for example, spline interpolation, which is further discussed in detail below.

After received signals are analyzed, and precise heartbeat times have been determined (for example, over a sample time of 10 seconds), a heart rate in beats per minute can be determined.

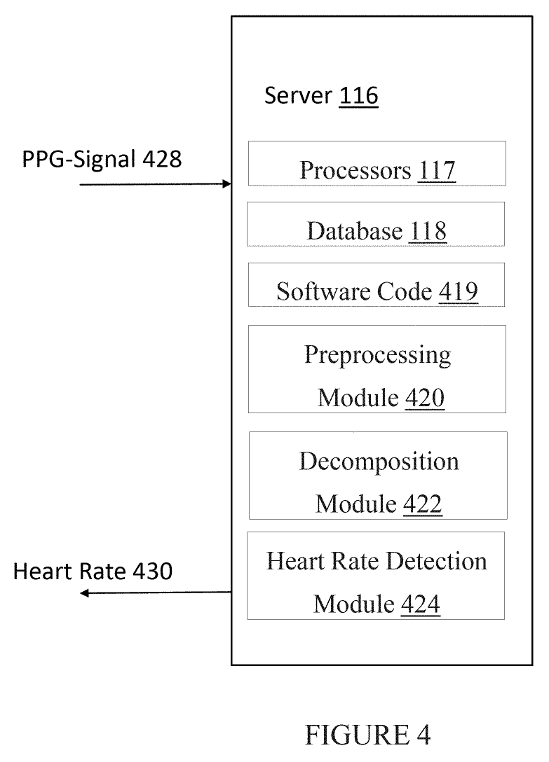

Referring now to FIG. 4 in conjunction with FIG. 1, FIG. 4 illustrates an exemplary implementation of server 116 of FIG. 1. As shown in FIG. 4, server 116 includes processors 117, database 118, software code 419, preprocessing module 420, decomposition module 422, and heart rate detection module 424. As further shown in FIG. 4, server 116 may be configured to receive PPG-signal 428 and transmit heart rate 430 through network 120 to any of the components of system 100. As noted above, the concepts described herein may be implemented on server 116, wearable device 108, communication device 106, other elements of system 100, or combinations thereof.

In other exemplary implementations, module components of server 117 (modules 420, 422, 424) may be implemented in hardware (including, for example, FPGAs and ASICs), firmware, software, and/or combinations thereof. As used herein, the term “module” is not meant as limiting to a specific physical form. Based on the particular application, modules can be implemented as firmware, software, hardware, and/or combinations of these. In an exemplary implementation, the modules may be implemented as dedicated circuitry (e.g., part of an ASIC). Doing so achieves lower power consumption with increased speed. In another exemplary implementation, the modules may be implemented as software, which runs on digital signal processors and/or general-purpose processors. Various combinations may be implemented. Furthermore, different modules can share common components or be implemented by the same components. There may or may not be a clear boundary between each module component. Finally, the methods described herein do not necessarily need to be implemented by modules at all. The modular structures described herein are provided simply as potential implementations and examples to facilitate the description.

Depending on the form of the modules, the “communication” between modules may also take different forms. Dedicated circuitry can be coupled to each other by hardwiring or by accessing a common register or memory location, for example. Software “communication” can occur by any number of ways to pass information between modules (or between software and hardware, if that is the case). The term “in communication” is meant to include all of these and is not meant to be limited to a hardwired permanent connection between two components. In addition, there may be intervening elements. For example, when two elements are described as being “in communication”, this does not imply that the elements are directly coupled to each other nor does it preclude the use of other elements between the two.

In an exemplary implementation, server 116 may be configured for receiving, by processors 417, photoplethysmographic (PPG) signal 428 communicated by a PPG sensor of wearable device 108 worn by a subject. Server 116 may also be configured for processing at least a portion of PPG-signal 428 through frequency band filters to create band outputs corresponding to heartbeat interval ranges. Sever 116 may be further configured for utilizing an indicated band output among the band outputs to determine a first heartbeat and a second heartbeat. In addition, server 116 may be configured for determining heart rate 430 based on at least the first heartbeat and the second heartbeat and causing heart rate 430 to be transmitted to at least wearable device 108.

In an exemplary implementation, after receiving PPG-signal 428, processor 117 may be configured to execute software code 419 in order to detect the presence of noise in PPG-signal 428 and carry out the exemplary implementations described herein. PPG-signal 428 may include a raw signal that may or may not have noise and artifact. By detecting the presence of noise in PPG-signal 428, redundant or unnecessary noise-removal operations may be avoided, thereby achieving faster, more efficient heart rate determinations.

In an exemplary implementation, detecting the presence of noise and artifact may include determining an input instantaneous-amplitude estimate of PPG-signal 428 and forming a time-smoothed version of PPG-signal 428. The instantaneous-peak-amplitude of the dominant component of PPG-signal 428 tends to be stable from heartbeat-to-heartbeat when the signal is free of noise. However, in the presence of noise, the instantaneous amplitude tends to fluctuate significantly. Accordingly, large deviations of the time-smoothed version of PPG-signal 428 may correspond to bursts of noise in PPG-signal 428.

In an exemplary implementation, processor 117 may be configured to detect large instantaneous amplitude deviations of the time-smoothed version of PPG-signal 428. When such instantaneous amplitude deviations exceed a predetermined amount, processor 117 may be configured to determine that noise and artifact are present in PPG-Signal 428.

In another exemplary implementation, when instantaneous amplitude deviations of the time-smoothed PPG-signal 428 are less than a predetermined amount, processor 117 may be configured to determine that noise and artifact are not present in PPG-Signal 428. When PPG-signal 428 is deemed to be free of noise, an exemplary implementation may include not performing preprocessing of PPG-signal 428 for noise removal, as discussed further below.

In yet another exemplary implementation, the instantaneous amplitude of PPG-Signal 428 is compared to a moving average of the instantaneous amplitude. For example, a moving average over 30 seconds of PPG-signal 428. If the instantaneous amplitude is greater than the moving average by predetermined multiplicative factor, the portion of PPG-signal 428 is flagged for noise. In one exemplary implementation the predetermined multiplicative factor can be a factor of 1.5. In other exemplary implementations, the predetermined multiplicative factor may be greater than 1.5 and/or less than 2.

In one exemplary implementation, upon detected large instantaneous amplitude deviations as discussed above, in order to ensure complete coverage of a noisy portion of PPG-signal 428, preprocessing module 420 may be configured to extend the duration of PPG-signal 428 that has been flagged for noise.

While utilizing the time-smoothed version of PPG-signal 428 for determining instantaneous amplitude deviations of PPG-signal 428 is especially well suited for detecting the presence of noise, other methods of determining the presence of noise in PPG-signal 428 may be utilized without diverting from the scope and spirit of the exemplary implementations described herein.

After processing PPG-signal 428 for noise detection as described above, server 116, via preprocessing module 420, may be configured to perform pre-processing of PPG-signal 428.

In an exemplary implementation, preprocessing module 420 may be configured to receive PPG-signal 428 and perform preprocessing of PPG-signal 428. In one exemplary implementation, preprocessing of PPG-signal 428 may include removing high-frequency noise and low-frequency artifact. Preprocessing may also include removing gross end effects including noise pedestal, baseline wander, and DC offset in PPG-signal 428.

In one exemplary implementation, processing PPG-signal 428 by preprocessing module 420 may include first removing gross end effects due to large slowly time-varying DC noise pedestal inherent in PPG-signal 428. This may be achieved by implementing basic straight-line de-trending techniques. Basic straight-line de-trending may function to mitigate end effects of PPG-signal 428 due to large DC offset occurring at the ends of raw input signals, i.e., occurring at the ends of a portion of PPG-signal 428.

In one exemplary implementation, removing gross end effect may further include removing pedestal at each end of at least a portion of the PPG-signal 428. In an exemplary implementation, pedestal at each end of at least a portion of PPG-signal 428 may be estimated by taking the mean of PPG-signal 428 signal vector over nominally 2 seconds from each end. A trend-line may then be implemented from the first sample time to the end sample time of PPG-signal 428. The trend-line thus passes through the mean values corresponding to the first sample time and the end sample time. Subtracting the values resulting from this trend-line computation from the raw input signal, i.e., PPG-signal 428, results in the de-trended output signal, i.e., a filtered PPG-signal 428.

In an exemplary implementation, removing large artifacts may include implementing high-pass and low-pass filtering to suppress large artifacts. High-pass and low-pass filtering may include implementing cascaded low-pass and high-pass filters to suppress high and low frequency artifacts, respectively. In another exemplary implementation, the high-pass filter may be implemented by subtracting the delay centered output of an internal low-pass filter from the input of the low-pass filter.

In an exemplary implementation, preprocessing filtering may be implemented utilizing linear-phase filters in order to preserve the primary morphological features of the underlying PPG-signal 428 and to align time delays from input to output at all frequencies. In another exemplary implementation, preprocessing filtering may include utilizing cascades of boxcar filters. In one exemplary implementation, preprocessing PPG-signal 428 may include utilizing a bandpass filter with corner frequencies at band pass high 0.5 hertz and bandpass low 10 hertz.

In another exemplary implementation, PPG-signal 428 may undergo further input processing to remove large artifacts existing outside the desired bandwidths of PPG-signal 428. In yet another exemplary implementation, other filter protocols for removing noise and artifact may utilized, e.g., blind source separation utilizing independent component analysis for uncovering independent source signal components and deducing linear mixtures of underlying sources. Other noise removing filtering processes may be implemented without diverting from the scope and spirit of the present implementations described herein, and have been fully contemplated.

In one exemplary implementation, in order to remove intrinsic latency delay associated with preprocessing PPG-signal 428, the output signal of preprocessing module 420, i.e., filtered PPG-signal 428, may be the same length as the input signal, i.e., raw PPG-signal 428, and aligned in time as well. In another exemplary implementation, the output signal may not be the same length as the input signal the filtered PPG-signal may not be time aligned. While the preprocessing methods described above are especially well suited for implementing the exemplary implementations described herein, other methods of separating time-series signals into composite sub-components using a signal reference may be implemented without diverting from the scope and spirit of the present implementations, and have been fully contemplated herein.

As used herein, the filtered PPG-signal 428 may be referred to as simply PPG-signal 428. Any reference to filtered PPG-signal 428 or simply PPG-signal 428 may thus refer to a filtered or non-filtered PPG-signal and should not be construed as limiting the PPG-signal 428 to a particular implementation.

In an exemplary implementation, after performing the preprocessing of PPG-signal 428, preprocessing module 420 may be configured to communicate filtered PPG-signal 428 to decomposition module 422. Decomposition module 422 may then perform operations on the filtered PPG-signal 428 in preparation for detecting heartbeats and determining heart rate.

Decomposition module 422 may be configured to decompose PPG-signal 428. Decomposition may be implemented in linear-phase to preserve morphology characteristics of PPG-signal 428. Decomposing PPG-signal 428 may also include separating PPG-signal 428 into a series of sub-signals that in aggregate substantially comprise PPG-signal 428 (substantially meaning the difference may be negligible). Accordingly, decomposition module 422 may be utilized for decomposing PPG-signal 428 by separating, from PPG-signal 428, a set of PPG sub-signals.

Decomposing PPG-signal 428 may include processing at least a portion of PPG-signal 428 through frequency band filters. In one exemplary implementation, the frequency bands filters may correspond to bandwidth ranges of 0.55-1.37 hertz, 0.78-2.33 hertz, and 1.26-4.25 hertz, respectively In another exemplary implementation, the frequency band filters are designed to be less than one octave wide. Stated another way, the bandwidth of the frequency band filters are chosen to prevent inclusion of two or more successive harmonics of the PPG-signal. In this manner, only the first harmonic of input signals substantially appears at the output for locally periodic input signals within the frequency band range. Consequently, at the time surrounding each heartbeat, the frequency band filter having a passband (i.e., bandwidth range) that covers the local fundamental pulse rate will be excited by the greatest amount, and therefore, will exhibit the greatest amplitude at the output.

In addition to having the largest amplitude, the largest amplitude output band of the frequency band filters will also exhibit the greatest purity of the local sinusoid since only the fundamental harmonic appears at the output of the frequency band filters. Accordingly, the frequency band output having the largest amplitude corresponds to the fundamental frequency of the input signal (i.e., the main component of the input signal).

In an exemplary implementation, frequency band filters may include a wavelet bank utilizing successively increasing semi-dyadic cascade of low-pass/high-pass separations. In another exemplary implementation, decomposition module 422 may be configured for decomposing PPG-signal 428 through the wavelet bank and producing an output. Wavelet bank outputs (i.e., frequency band outputs), may be delay-aligned and centered in time upon the input signal, PPG-signal 428. In this manner, the intrinsic delay due to latency associated with the wavelet bank may be removed in order to ensure accurate heart rate determinations. Accordingly, processing at least a portion of PPG-signal 428 through frequency band filters may include creating band outputs.

In one exemplary implementation, decomposing PPG-signal 428 may include processing PPG-signal 428 through frequency band filters to create band outputs corresponding to a plurality of heartbeat interval ranges. Heartbeat interval ranges may correspond to frequency ranges associated with expected heart rates of the subject. For example, heartbeat interval ranges may correspond to a normal range, a tachycardia range, and a bradycardia range. In other implementations, the heartbeat interval ranges may correspond to different ranges that may be useful in separating PPG-signal 428 into frequency ranges corresponding to likely heart rate ranges of the user.

Further signal processing techniques may be implemented for ensuring that the frequency band filters produce outputs that are robust and free of signal degradation complexities. For example, in one exemplary implementation, utilizing frequency band filters may include applying an input vector gain between each of the successive cascades in order to address signal attenuation and other signal degradation issues. Various other approaches may be utilized for filtering PPG-signal 428 including: matched filters, second order derivatives, nonlinear timescale decomposition, adaptive filtering, dynamic time warping, artificial neural networks, or hidden Markov models.

Decomposition module 422 may be configured to communicate decomposed portions of PPG-signal 428 (e.g., frequency band outputs) to heart rate detection module 424. In an exemplary implementation, heart rate detection module 424 may be configured for tracking each of the communicated frequency band outputs and determining, by comparison, which of the frequency band outputs has the largest amplitude. The largest amplitude band output may be indicative of the user's heart rate being within that band and that band may thus be used to determine heartbeats. Thus, heart rate detection module 424 may be configured for determining amplitudes for the band outputs and determining the largest amplitude band output among the band outputs, where identification of an “indicated” band output corresponds to the largest amplitude band output. The indicated band output may then be utilized to determine a first heartbeat, a second heartbeat, and a heart rate from PPG-signal 428, as discussed further below.

In another implementation, heart rate detection module 424 may be configured to utilize more than one frequency band output. For example, heartrate detection module 424 may generate a running estimate of the amplitudes of the frequency band outputs and may perform further processing on the band outputs with the two highest amplitudes. This method may provide faster heart rate determination/tracking in cases where heart rates are changing rapidly beat-to-beat and perhaps moving from one frequency band to another. Accordingly, in an exemplary implementation, in addition to determining the largest amplitude band output, analysis module 424 may be configured for determining a second largest amplitude band output and utilizing the second largest amplitude band output to determine an “anticipated” heartbeat. The “anticipated” heartbeat may potentially be the appropriate heartbeat to analyze if the heart rate has moved into the new frequency band. Heart rate detection module 424 may thus be further configured for utilizing the anticipated heartbeat in determining heart rate 430 when the indicated band output changes, to provide faster heart rate tracking and transmission to wearable device 108.

For example, in one exemplary implementation, the largest amplitude band may correspond to the normal band range, while the second largest amplitude band output may correspond to the tachycardia heart rate range. Tracking both band outputs may then provide for faster tracking in the case where heart rate is changing rapidly, as can occur with certain arrhythmias such as atrial fibrillation.

In one implementation, heart rate detection module 424 may be configured to “qualify” the second largest amplitude band for further processing (e.g., the determining of anticipated heartbeats). Qualifying the second largest amplitude band output may include, for example, determining if the second largest amplitude band output is a sufficient fraction of the largest amplitude band output. In the case where the second largest amplitude output is a sufficiently small fraction (e.g., <0.85), heart rate detection module 424 may forgo further processing of the second largest amplitude output band and the determination of anticipated heartbeats in that band.

Heart rate detection module 424 may be further configured to utilize the band outputs described above in determining heartbeats (or, similarly, anticipated heartbeats). Determining heartbeats in this sense generally refers to determining the times at which heartbeats take place. Such times may then be used in determining heart rates.

One technique for determining heartbeats contemplated by the present disclosure utilizes the rate of change of the PPG-signal (also referred to as the PPG-signal gradient). FIG. 5 depicts an exemplary PPG-signal 428 (having a first PPG-signal peak 502 and a second PPG-signal peak 504), and a PPG-signal gradient 506 (having maximum gradients 508A, 508B, and 508C).

In one exemplary implementation, determining the first heartbeat and a second heartbeat may include determining the first heartbeat and second heartbeat from maximum gradients 508 of PPG-signal 526.

As shown in FIG. 5, maximum gradients 508 depict the locations of the fastest incline of the PPG-signal 528, i.e., the maximum values of PPG-signal gradient 506. These locations can then be deemed heartbeats (i.e., the times at which heartbeats occur). The heartbeats may then be utilized to determine heart rate.

Sampling rate limitations inherent in the acquisition of PPG-Signal 428 can result in the PPG-signal peaks (502 and 504) and gradient peaks (508A,B,C) shown in FIG. 5 appearing to be in a location different from the actual peak (as would be depicted should there be no limit on sampling rate).

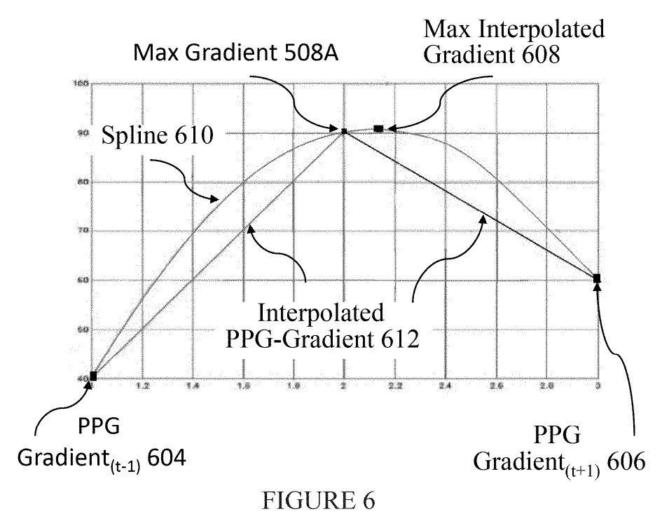

In order to overcome the sampling resolution limitations inherent in the PPG sensing process and to increase the accuracy of heart rate determinations, heart rate detection module 424 may be configured to utilize various methods for determining a more accurate gradient peak. One such method may be explained with reference to FIG. 6. FIG. 6 depicts maximum gradient point 508A from FIG. 5, along with two other gradient curve points (depicted as PPG gradient_{T-1} 604 and PPG gradient_{T+1} 606) that can be used with, e.g., a spline interpolation 610 to determine a more accurate gradient peak 608. Accordingly, heart rate detection module 424 may be configured for determining a heartbeat utilizing maximum gradient 508A, at least two other data points from the gradient of PPG-signal 428, and a mathematical method.

In an exemplary implementation, the mathematical model may comprise spline interpolation. Also, the at least two other points may include samples of PPG-signal gradient 506 at immediate intervals before and after maximum gradient 508, i.e., PPG gradient_{T-1} 604 and PPG gradient_{T+1} 606. As further shown in FIG. 6, the more accurate gradient peak 608 may be determined by utilizing the three points (gradient_{T-1} 604, PPG gradient_{T+1} 606, and maximum gradient 508A) and spline interpolation, or another method, thus increasing the accuracy of heart rate determinations.

In one implementation, a state based sequence detector may be utilized to determine the two other data points from the gradient of the PPG-signal and to implement the mathematical method. While a state based sequence detector is especially well-suited for implementing the methods described herein for determining the other data points and implementing the mathematical method, other implementations may be utilized without diverting from the scope and spirit of the implementations described herein.

The processes described above can be repeated for other intervals containing a heartbeat in PPG-signal 428 and the time intervals between heartbeats allow heart rate 430 to be determined. Accordingly, heart rate detection module 424 may be configured to utilize the exemplary processes described above for determining heart rate 430 based on at least a first heartbeat and a second heartbeat and to cause heart rate 430 to be transmitted to at least wearable device 108 and/or any other component of system 100.

In an alternative heartbeat determination method, PPG-signal peaks are utilized instead of gradient peaks. For example, heart rate detection module 424 may configured to utilize frequency band output signals for determining a first PPG-signal peak 502 and second PPG-signal peak 504 from at least a portion of PPG-signal 428 and then determining a first heartbeat and a second heartbeat from the first PPG-signal peak 502 and the second PPG-signal peak 504. For example, the heartbeat may be considered to be at the location of the PPG-signal peak, or at an offset from the peak. The heart rate detection module 424 may then determine heart rate 430 based on the first heartbeat and the second heartbeat.

In order to overcome the sampling resolution limitations inherent in the PPG sensing process and to increase the accuracy of heart rate determinations, heart rate detection module 424 may be configured to utilize various methods for determining more accurate PPG-signal peaks. One such method may be explained as similar to the process described above with reference to FIG. 6. In a similar vein, heart rate detection module 424 may be configured for determining a heartbeat utilizing PPG-signal peak 502, at least two other data points from PPG-signal 428, and a mathematical method.

In an exemplary implementation, the mathematical model may comprise spline interpolation, but other methods may be utilized, as noted above.

The at least two other points from the PPG-signal 428 may, in one implementation, include a positive-going zero crossing and a negative-going zero crossing nearest PPG-signal peak 502. Such PPG-signal zero-crossings may be determined using a zero-crossing narrowband filter. The zero-crossing narrowband filter may be included in the frequency band filters as described above. The narrowband nature of the zero-crossing filter tends to produce output waveforms that are, on a time local per cycle basis, close to sinusoidal in waveform. Because these waveforms are substantially sinusoidal, they produce zero-crossings that are well behaved and reliable. The zero-crossing narrowband component of PPG-signal 428, is time aligned with PPG-signal 428. In this manner, the zero-crossings function to bracket PPG-signal peaks 502,504 in PPG-signal 428. A more accurate PPG-signal peak may then be determined utilizing the zero crossings with the original PPG-signal peak and a mathematical method, as described above.

In one implementation, a state based sequence detector may be utilized to determine the two other data points from the PPG-signal and to implement the mathematical method. While a state based sequence detector is especially well-suited for implementing the methods described herein for determining the other data points and implementing the mathematical method, other implementations may be utilized without diverting from the scope and spirit of the implementations described herein.

As discussed, the PPG-signal peaks, or the more accurate PPG-signal peaks, may be used to identify heartbeats at the locations of the peaks, or at some offset therefrom. These heartbeats may then be used to determine a user's heart rate, which may be, for example transmitted to and displayed on wearable device 108 or any other component of system 100.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also contemplated that may include one or more processors and one or more memories coupled to the one or more processors. A memory, which can include a computer-readable storage medium, may include, encode, store, or the like, one or more programs that cause one or more processors to perform one or more of the operations described herein. Computer implemented methods consistent with one or more implementations of the current subject matter can be implemented by one or more data processors residing in a single computing system or across multiple computing systems. Such multiple computing systems can be connected and can exchange data and/or commands or other instructions or the like via one or more connections, including but not limited to a connection over a network (e.g., the internet, a wireless wide area network, a local area network, a wide area network, a wired network, or the like), via a direct connection between one or more of the multiple computing systems, etc.

While certain features of the currently disclosed subject matter are described for illustrative purposes in relation to particular implementations, it should be readily understood that such features are not intended to be limiting. The claims that follow this disclosure are intended to define the scope of the protected subject matter.

Although a few embodiments have been described in detail above, other modifications are possible. For example, the method steps described herein do not require the particular order shown, or sequential order, to achieve desirable results.

The present disclosure contemplates that the calculations disclosed in the embodiments herein may be performed in a number of ways, applying the same concepts taught herein, and that such calculations are equivalent to the embodiments disclosed.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” (or “computer readable medium”) refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” (or “computer readable signal”) refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, computer programs and/or articles depending on the desired configuration. Any methods or the logic flows depicted in the accompanying Figures and/or described herein do not necessarily require the particular order shown, or sequential order, to achieve desirable results. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. The implementations described above can be directed to various combinations and subcombinations of the disclosed features and/or combinations and subcombinations of further features noted above. Furthermore, above described advantages are not intended to limit the application of any issued claims to processes and structures accomplishing any or all of the advantages.

Additionally, section headings shall not limit or characterize the invention(s) set out in any claims that may issue from this disclosure. Specifically, and by way of example, although the headings refer to a “Technical Field,” such claims should not be limited by the language chosen under this heading to describe the so-called technical field. Further, the description of a technology in the “Background” is not to be construed as an admission that technology is prior art to any invention(s) in this disclosure. Neither is the “Summary” to be considered as a characterization of the invention(s) set forth in issued claims. Furthermore, any reference to this disclosure in general or use of the word “invention” in the singular is not intended to imply any limitation on the scope of the claims set forth below. Multiple inventions may be set forth according to the limitations of the multiple claims issuing from this disclosure, and such claims accordingly define the invention(s), and their equivalents, that are protected thereby.

What is claimed is:

1. A computer program product comprising a non-transitory, machine-readable medium storing instructions which, when executed by at least one programmable processor, caused at least one programmable processor to perform operations comprising:
receiving, at the at least one programmable processor, a photoplethysmographic (PPG) signal communicated by a PPG sensor of a wearable device worn by a subject;
determining a first heartbeat and a second heartbeat from a maximum gradient of the PPG-signal;
determining a heart rate based on at least the first heartbeat and the second heartbeat; and
causing the heart rate to be transmitted to at least the wearable device.

2. The computer program product of claim 1, wherein determining the first heartbeat further comprises utilizing the maximum gradient, at least two other data points from a gradient of the PPG-signal, and a mathematical method.

3. The computer program product of claim 2, wherein the two other data points comprise a first PPG gradient and a second PPG gradient.

4. The computer program product of claim 2, wherein the mathematical method comprises spline interpolation.

5. The computer program product of claim 2, wherein a state based sequence detector is utilized to determine the two other data points from the gradient of the PPG-signal and to implement the mathematical method.

6. A computer program product comprising a non-transitory, machine-readable medium storing instructions which, when executed by at least one programmable processor, caused at least one programmable processor to perform operations comprising:
receiving, at the at least one programmable processor, a photoplethysmographic (PPG) signal communicated by a PPG sensor of a wearable device worn by a subject;
determining a first PPG-signal peak and a second PPG-signal peak from at least a portion of the PPG-signal;
determining a first heartbeat and a second heartbeat from the first PPG-signal peak and the second PPG-signal peak;
determining a heart rate based on at least the first heartbeat and the second heartbeat; and
causing the heart rate to be transmitted to at least the wearable device.

7. The computer program product of claim 6, wherein determining the first heartbeat further comprises utilizing the first PPG-signal peak, at least two other data points from the PPG-signal, and a mathematical method.

8. The computer program product of claim 6, wherein the two other data points from the PPG-signal are a positive-going zero crossing and a negative-going zero crossing nearest the PPG-signal peak.

9. The computer program product of claim 7, wherein the mathematical method comprises spline interpolation.

10. The computer program product of claim 7, wherein a state based sequence detector is utilized to determine the two other data points from the PPG-signal and to implement the mathematical method.

11. A computer program product comprising a non-transitory, machine-readable medium storing instructions which, when executed by at least one programmable processor, caused at least one programmable processor to perform operations comprising:
receiving, at the at least one programmable processor, a photoplethysmographic (PPG) signal communicated by a PPG sensor of a wearable device worn by a subject;
processing at least a portion of the PPG-signal through a plurality of frequency band filters to create a plurality of band outputs corresponding to a plurality of heartbeat interval ranges;
utilizing an indicated band output among the plurality of band outputs to determine a first heartbeat and a second heartbeat;
determining a heart rate based on at least the first heartbeat and the second heartbeat; and
causing the heart rate to be transmitted to at least the wearable device.

12. The computer program product of claim 11, wherein the plurality of heartbeat interval ranges correspond to at least a normal range, a tachycardia range, and a bradycardia range.

13. The computer program product of claim 11, wherein a bandwidth of at least one of the plurality of frequency band filters is chosen to prevent inclusion of two or more successive harmonics of the PPG-signal.

14. The computer program product of claim 11, wherein processing the portion of the PPG-signal through the plurality of frequency band filters further comprises utilizing a plurality of successively increasing semi-dyadic cascade of low-pass/high-pass separations.

15. The computer program product of claim 11, further comprising determining a plurality of amplitudes for the plurality of band outputs and determining a largest amplitude band output among the plurality of band outputs,
wherein the indicated band output corresponds to the largest amplitude band output.

16. The computer program product of claim 15, wherein determining the first heartbeat comprises:
determining a first PPG-signal peak of the indicated band output; and
determining the first heartbeat utilizing the first PPG-signal peak, at least two other data points from the PPG-signal, and a mathematical method.

17. The computer program product of claim 16, wherein the two other data points comprise a positive-going zero crossing and a negative-going zero crossing nearest to the first PPG-signal peak.

18. The computer program product of claim 16, wherein a state based sequence detector is utilized to determine the two other data points from the PPG-signal and to implement the mathematical method.

19. The computer program product of claim 16, wherein the mathematical method is spline interpolation.

20. The computer program product of claim 15, the operations further comprising:
determining a second largest amplitude band output; and
utilizing the second largest amplitude band output to determine an anticipated heartbeat.

21. The computer program product of claim 20, wherein determining the anticipated heartbeat comprises:
determining a PPG-signal peak of the second largest amplitude band output; and
determining the anticipated heartbeat utilizing the PPG-signal peak of the second largest amplitude band output, at least two other data points from the second largest amplitude band output, and a mathematical method.

22. The computer program product of claim 21, wherein the two other data points comprise a positive-going zero crossing and a negative-going zero crossing nearest to the PPG-signal peak of the second largest amplitude band output.

23. The computer program product of claim 21, wherein the mathematical method is spline interpolation.

24. The computer program product of claim 20, the operations further comprising utilizing the first anticipated heartbeat in determining the heart rate when the indicated band output changes, to provide faster heart rate tracking and transmission to the wearable device.
